(19) United States
(12) Patent Application Publication
(10) Pub. No.: US 2013/0204243 A1
(43) Pub. Date: Aug. 8, 2013

(54) HANDHELD ELECTROSURGICAL GENERATOR

(71) Applicant: John Jordan Newkirk, Evergreen, CO (US)

(72) Inventor: John Jordan Newkirk, Evergreen, CO (US)

(21) Appl. No.: 13/751,557

(22) Filed: Jan. 28, 2013

Related U.S. Application Data
(60) Provisional application No. 61/594,087, filed on Feb. 2, 2012.

(51) Int. Cl. A61B 18/12

(52) U.S. Cl. CPC A61B 18/1206
USPC 606/34

(57) ABSTRACT

A handheld electrosurgical generator including a pen, internal controller, signal generator, and RF amplifier. The pen electro-mechanically connects to an electrosurgical needle. The controller is embedded within the pen to control the RF output at the electrosurgical needle tip. An electrosurgical system incorporates the handheld electrosurgical generator along with at least one of a data processing unit and a data entry point in communication with the controller of the handheld electrosurgical generator.

### PRIORITY CLAIM

This application claims priority to U.S. Provisional Patent Application No. 61/594,087 titled “Handheld Electrosurgical Generator” of John J. Newkirk filed on Feb. 2, 2012, and is related to U.S. patent application Ser. No. 13/585,014 titled “Intelligent electrosurgical electrode and tracking system” of John J. Newkirk filed on Aug. 14, 2012 which claims the benefit of U.S. Provisional Patent Application No. 61/525,134 titled “Traceable Electrode And Tracking System For Single-Use Surgical Needles” of John J. Newkirk filed on Aug. 18, 2011, each of which is hereby incorporated by reference as though fully set forth herein.

### BACKGROUND

Electrosurgery is a form of surgery in which body tissue is cut or cauterized by a high frequency current, and offers certain advantages to conventional knife dissection. A variety of electrosurgical tools have been employed. The electrosurgical electrode has been used for many years in a wide variety of applications, e.g., for delicate cutting, cauterization, and normal surgical cutting as with a scalpel.

When in a cutting mode, the current created when the electrosurgical electrode touches the body tissue incises the tissue. By varying the mode of the RF generator output, it is also possible to utilize the electrosurgical device to enhance cauterization or coagulation of blood in a wound. In the cauterizing mode, the electrosurgical electrode generates much more heat than when in the cutting mode.

Electrosurgical electrodes may be composed of stainless steel, although some are composed of other alloys such as those containing primarily tungsten, molybdenum, chromium, nickel or cobalt. More recent electrodes implement an ultra-sharp refractory alloy. Such ultra-sharp electrodes (also known as “electrosurgery needles”) have been specifically designed for consistency, symmetry, sharpness and tapering to produce superior results. For example, use of an ultra-sharp tungsten needle allows for significantly reduced RF power due to the concentration of energy at the ultra-sharp tip. Efficient cutting at lower power reduces blood loss and leads to much cleaner and less traumatic cuts, resulting in less scar tissue. Use of the ultra-sharp needle also eliminates the drag when cutting tissue. This “no-touch” technique allows the surgeon a sensitive “feel”, which is a significant benefit when performing extreme microsurgery. When used in the cauterization mode, the ultra-sharp electrode may again be used at relatively lower RF power, thereby eliminating problems associated with excessive heat, such as accidental burns to the patient and/or melting the electrode tip, and with greater control over the direction and location of sparking.

Electrosurgical electrodes are typically used with bulky AC-powered electrosurgical generators. In addition to being expensive, these generators often have to be wheeled around on large carts, or remain in a dedicated space with multiple wires connected to the patient and/or wall outlets.

### BRIEF DESCRIPTION OF THE DRAWINGS

FIG. 1 shows an example handheld electrosurgical generator, wherein (a) is a side plan view, and (b) is a top plan view.

FIG. 2 is an enlarged view of an example mechanical interconnect for the needle assembly shown in (a) an unassembled configuration, and (b) an assembled configuration.

FIG. 3 is a top plan view of an external power supply/return electrode embodied as a body patch.

FIG. 4 is a high-level block diagram of an example control circuit which may be provided in the handheld electrosurgical generator.

FIG. 5 shows the handheld electrosurgical generator as it may be implemented as part of an electrosurgical system.

FIG. 6 shows another example handheld electrosurgical generator with a removable “snap-in” electrode, wherein (a) is a side plan view, (b) is a top plan view, (c) is a bottom plan view, (d) is cross-sectional view, and (e) is a perspective view.

FIG. 7 shows the pen/handle of the handheld electrosurgical generator illustrated in FIG. 6, wherein (a) is a side plan view, (b) is a top plan view, (c) is a bottom plan view without an electrode inserted.

### DETAILED DESCRIPTION

A handheld electrosurgical generator is disclosed, for example as it may be provided as a unit together with an integral power supply. In an example, the electrosurgical generator may be implemented as either a monopolar or bipolar generator. The power supply may be housed entirely within a pen and/or as part of a disposable electrosurgical grounding pad/power supply. In any event, the handheld electrosurgical generator eliminates the need for the bulky AC-powered electrosurgical units commonly used today. Such miniaturization is made feasible by dramatically reduced power requirements of the ultra-sharp tungsten electrode disclosed by Newkirk & Manwaring (U.S. Pat. No. 4,927,420) and U.S. patent application Ser. No. 13/585,014 (Newkirk) cited above.

The following definitions are used herein when referring to electrode configurations and circuit topologies used in electrosurgery. In a “monopolar” configuration the patient is attached to a return electrode (a relatively large metal plate or a flexible metalized plastic pad connected to the return electrode of the RF generator). The surgeon uses an electrosurgical needle to make contact with the tissue. In a “bipolar” configuration the voltage is applied to the patient using a pair of similarly sized electrodes or needles. For example, special forceps may be used, with one tine connected to one pole of the RF generator and the other tine connected to the other pole of the generator. When a piece of tissue is held by the forceps, RF current flows from one tine to the other tine of the forceps, heating the intervening tissue.

It is noted that as used herein, the terms “electrosurgery needle,” “electrosurgical needle,” “electrosurgical electrode,” and “electrode” apply to, but are not limited to, both monopolar and bipolar configurations.

The handheld electrosurgical generator may be simple for a user to operate, including one or a few controls (buttons/knobs/sliders) for power and waveform adjustment. An electronic bar graph (e.g., operated by LEDs) provides visual feedback. The handheld electrosurgical generator is operable with a variety of electrosurgical needles, including but not limited to so-called “single-use” needles.

The handheld electrosurgical generator includes a controller (e.g., a control circuit, RF amplifier, and/or microprocessor) embedded in the pen. The controller may be used to control output (e.g., creating electrical waveforms and pulses), and to provide feedback (e.g., via the electronic bar graph/LEDs and/or tactical and/or audio output) for the user. The controller may be updated using updatable firmware, for example, to provide new output (electrical waveforms and pulses) and/or power levels and to facilitate user feedback.

The controller may also enable product traceability to monitor use of the electrosurgical needles. Traceable electrodes are discussed in more detail in co-owned U.S. patent application Ser. No. 13/585,014 titled “Intelligent electrosurgical electrode and tracking system” of John J. Newkirk filed on Aug. 14, 2012, and U.S. Provisional Patent Application No. 61/594,087 titled “Handheld Electrosurgical Generator” of John J. Newkirk filed on Feb. 2, 2012, each of which is hereby incorporated by reference for all that it discloses as though fully set forth herein. In addition, the controller may enable encryption so that the handheld electrosurgical generator can only be used with certain types of electrosurgical needles.

Ultra-sharp electrosurgical needles give the surgeon unprecedented precision during surgery, resulting in the patient experiencing less bleeding, less scarring, and faster healing than standard methods of surgery. Although the cost of electrosurgical needle may at first seem high, the needle actually represents one of the lowest costs of surgical procedure. A recent study comparing laser surgery with electrosurgery revealed significant reductions in time, blood loss, and postoperative pain over the course of 20 typical bilateral reduction mammoplastys. See, e.g., Table 1:

TABLE 1
Laser versus Electrocautery

| | Laser | Electrocautery |
|---|---|---|
| Dissection Time | 61 minutes | 32 minutes |
| Blood loss | 176 cc | 72 cc |
| Est. Cost | $698 | $114 |

Source: Michigan State University

However, electrosurgical electrodes typically need to be used with bulky AC-powered electrosurgical generators. In addition to being expensive, these generators often have to be wheeled around on large carts, or remain in a dedicated space and require multiple electrical connections to the patient and/or external power.

A handheld electrosurgical generator is disclosed, for example as it may be provided as a unit together with an integral power supply. The handheld electrosurgical generator may be the size of a conventional soldering iron, and does not need to be operated in conjunction with an external generator. The power supply may be housed entirely within a pen and/or as part of a disposable electrosurgical grounding pad/power supply. In any event, the handheld electrosurgical generator is made possible by the dramatically reduced power required by the ultra-sharp tungsten tip described by Newkirk & Manwaring (U.S. Pat. No. 4,927,420) and U.S. patent application Ser. No. 13/585,014 (Newkirk) and eliminates the need for the bulky AC-powered electrosurgical units commonly used today.

Before continuing, it is noted that as used herein, the terms “includes” and “including” mean, but is not limited to, “includes” or “including” and “includes at least” or “including at least.” The term “based on” means “based on” and “based at least in part on.”

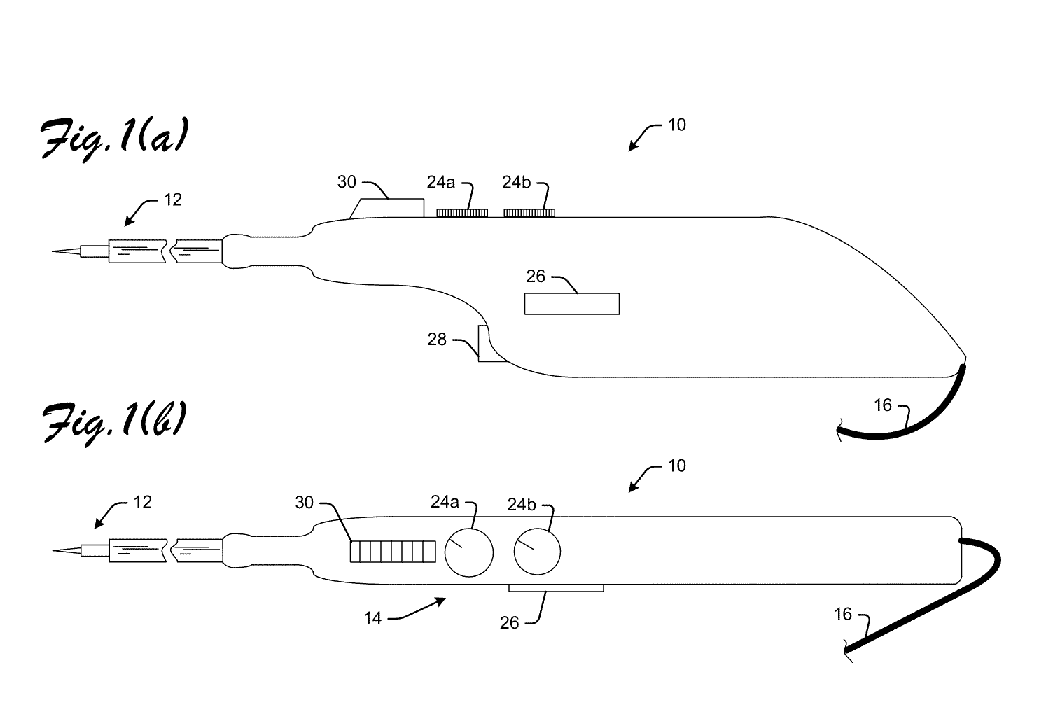

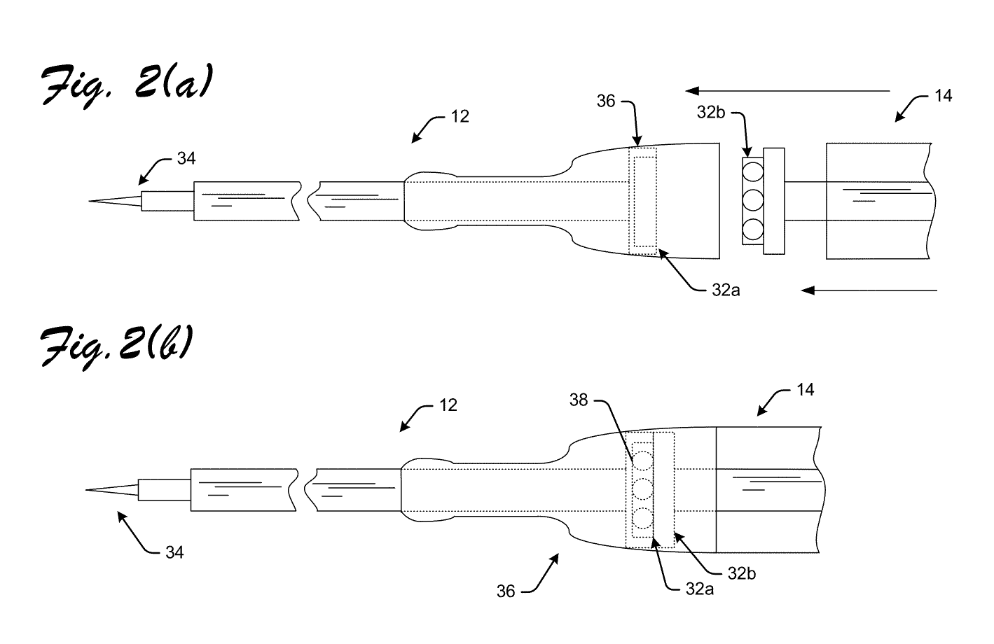

FIG. 1 shows an example handheld electrosurgical generator 10, wherein (a) is a side plan view, and (b) is a top plan view. The handheld electrosurgical generator 10 generally includes an electrosurgical needle/needle assembly 12 and a pen or handle 14 and may be provided as a unit together with an integral power supply (not shown). For example, the power supply may be a battery (or multiple batteries) housed entirely within the pen 14. The batteries may be rechargeable, and the handheld electrosurgical generator 10 may include a cord or cable 16 may be used, at least in part, for recharging the batteries. Alternatively, the batteries may be disposable. In another example, the power supply may be part of an external electrosurgical grounding pad/power supply 18 (see, e.g., FIG. 3), connected to the handheld electrosurgical generator 10 via the cable 16. The electrosurgical grounding pad/power supply 18 may be disposable, particularly in the case where the pad 18 is used on patients, and may include a battery 20, a pair of pad electrodes 22 a-b, and a backing 23. In yet another example the cable 16 could instead facilitate a connection to an electrical outlet (not shown).

The handheld electrosurgical generator 10 may be simple for a user to operate, including one or only a few multi-function controls (e.g., buttons/knobs/sliders) for power and waveform adjustment. By way of illustration, the handheld electrosurgical generator shown in FIG. 1 includes two control wheels 24 a-b and a power button 26. One control wheel 24a may be used to control power intensity to the electrosurgical needle 12, and the other control wheel 24b may be used for waveform adjustment. It is noted, however, that more than one function may be integrated into a single control. For example, a user may press a single control wheel (e.g., 24a) to switch between power intensity and waveform, and then turn the control wheel (e.g., 24a) to make the adjustment(s). It is to be understood that an adjustment of the waveform could come in the form of a pulse change (e.g., pulse length and/or peak intensity).

The handheld electrosurgical generator 10 is also shown as it may include a light 28 and display 30, such as an electronic bar graph (e.g., multiple multi-colored LEDs). The display 30 may provide visual feedback for the user. For example, the LED lights may include green, yellow, and red to indicate power intensity being delivered by the electrosurgical needle. Of course, any suitable output may be provided for the user, and in any suitable manner (e.g., audible and/or tactical output may also be output). The handheld electrosurgical generator 10 is operable with a variety of electrosurgical needles 12, including but not limited to so-called “single-use” needles.

The handheld electrosurgical generator 10 may be sterilized by autoclave, ETO, or other conventional sterilization methods. The handheld generator 10 may also be quasi-disposable, that is rated, for example, for several hundred autoclave cycles before certain sealing mechanisms should be refurbished.

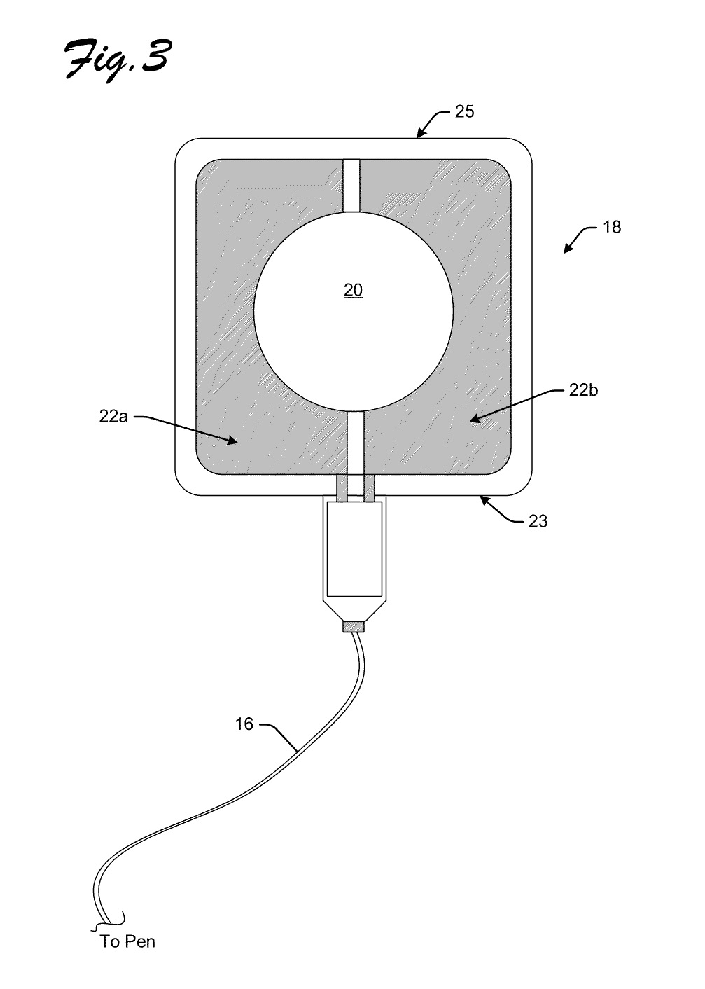

FIG. 2 is an enlarged view of an example mechanical interconnect 32 a-b for the electrosurgical needle 12 shown in (a) an unassembled configuration, and (b) an assembled configuration. An example electrosurgical needle 12 may include a needle tip 34 connected to a shroud 36 with a mechanical interconnect 32 a-b, which serves as an interlocking mechanism for attaching the needle 12 to the “pen” or handle 14. In an example, the needle 12 may have a tungsten or other refractory metal or alloy electrode needle tip 34 with a radius of about 50 microns or smaller (i.e., qualifying as an ultra-sharp tip as known in the art). Power is provided to the needle 12 via the pen 14. A mechanical interconnect 32 a-b is used to attach the needle 12 to the pen 14, and includes electrical contacts 38 to provide a connection (e.g., power and/or data) between the pen 14 and the needle 12. In an example, the electrical contacts 38 may include a power line, and a ground line, although other configurations are also possible. Data input/output lines, as part of the electrical contacts 38, may also be provided, e.g., when implemented with a traceable electrode and tracking system.

It is noted that the electrical/mechanical interconnect 32 a-b shown and described herein is an example of a suitable interconnect which may be used. But the interconnect 32 a-b shown and described is illustrative and not intended to be limiting. Other interconnects now known or later developed may also be used, as will be readily understood by those having ordinary skill in the art after becoming familiar with the teachings here. For example, the connector may include ball bearings and pads or may include a male-female push-pull snap connector. Other contacts are also possible.

FIG. 3 is a top plan view of an external power supply 18 embodied as a body patch. The body patch power supply 18 can be the size of a conventional disposable grounding pad and/or the size of the battery 20 used therein. The body patch power supply 18 includes a peel-off backing 23 with gel and/or adhesive 25 (schematically pointed to in FIG. 3) on the underside thereof that can be used to adhere to a patient's body, and may further include a foil layer (not shown) to provide a ground. The body patch power supply 18 also includes at least one integrated battery 20 (e.g., one or more ultra thin cell(s) such as part no. CP505050 3.0V, 3000 mAh lithium manganese dioxide batteries commercially available from Guangzhou Markyn Battery Co., Ltd., China, and other batteries available from this and other manufacturers). It is to be understood that, alternatively, the battery 20 or other power supply could be carried within the pen 14 and be within the scope of the disclosure.

The integrated battery 20 may include a positive terminal electrode 22a on one side and a negative terminal electrode 22b on the opposite side. The negative side of the body patch power supply 18 can be affixed to the patient's body (not shown) using an adhesive and/or electrolytic gel to make a good grounding contact, and may also output 3-12V to the handheld electrosurgical generator 10. The body patch power supply 18 is connected to a handheld controller 10 via suitable cabling 16.

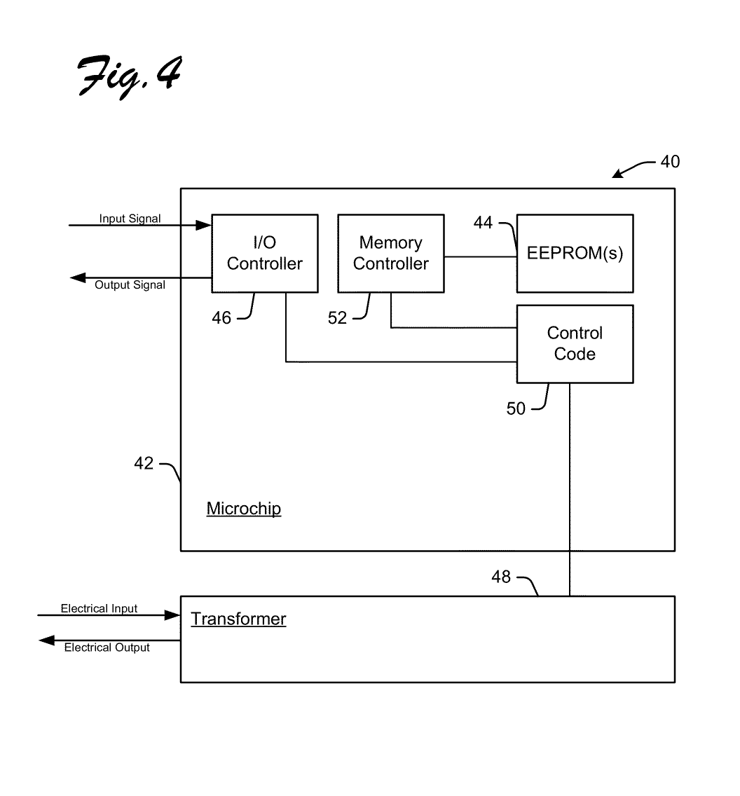

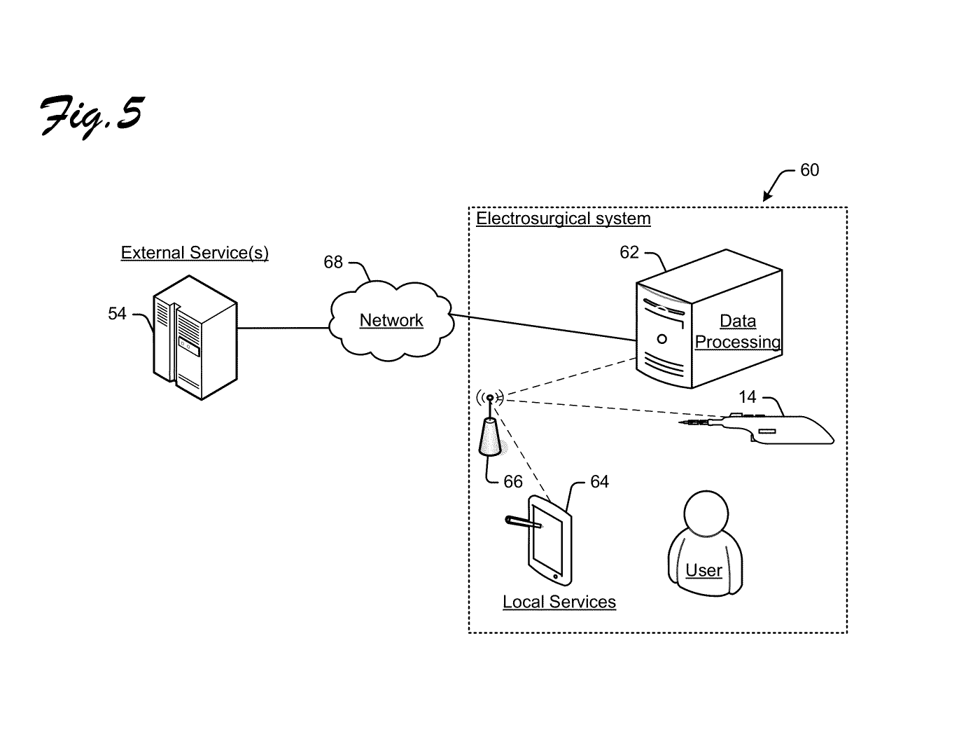

FIG. 4 is a high-level block diagram of an example main controller 40 (e.g., a control circuit, RF amplifier, and/or microprocessor) which may be provided or otherwise embedded in the handheld electrosurgical generator 10. For example, the main controller 40 may include a microchip 42 with at least one serial EEPROM 44, which, at least in part, receives power at the pen 14. The main controller 40 may be further provided with an I/O controller 46, which may be used to control output (e.g., creating electrical waveforms and pulses via an electrical transformer 48), and to provide feedback (e.g., via the electronic bar graph/LED display 30 and/or tactical and/or audio output) for the user. The main controller 40 may be further provided with a control code 50 that may be updated using updatable firmware, for example, to provide new output (e.g., electrical waveforms, pulses, and/or power) and user feedback.

A feedback circuit may also be provided. The feedback circuit may be implemented via the body patch and the control code 50 to provide for automatic or semi-automatic control of power level and/or waveform output by the electrosurgical needle. In an example, the feedback circuit is configured to sense the resistance of various types of tissue (fascia, muscle, fatty tissue, etc.) in contact with the electrode, deliver an electrical signal to the main controller 40, wherein the control code 50 processes the signal and automatically adjusts power levels and/or waveform for the type of tissue. The feedback circuit may also be used to respond to other predetermined conditions, for example, to shut off output power if a short circuit or insufficient grounding circuit is detected.

The microchip 42 may be used for other purposes, such as recording actual use. The microchip 42 may be provided with a memory controller 52 and may process data and/or record data and issue a data signal to an external processor 54 (as in FIG. 5) for further processing. External processing may include analyzing data and issuing the user alerts when appropriate.

The handheld electrosurgical generator 10 may also be used with traceable electrodes, and thus the main controller 40 may further be implemented/programmed with a tracking system. Further processing may also include correlating the data for the needle 12 to a serial number or other identifying indicia (not shown) for the needle 12. The data may be used, for example, to provide feedback to the user and/or the manufacturer, and to issue alerts when a needle 12 is used more than recommended. For example, a user may be notified when a single-use needle has been used more than once and/or when one or more performance parameters drop below a certain threshold.

FIG. 5 shows the handheld electrosurgical generator 10 as it may be implemented as part of an electrosurgical system 60. The handheld electrosurgical generator 10 may include and/or be operatively associated with various data processing and services (both local and/or remote), such as a data processing unit 62; local services 64, such as a data entry point or touch screen; and/or an external processor 54. These various data processing and services may be linked to one another and/or the handheld electrosurgical generator 10 via a wireless device 66, a network 68, a hard-wired connection (not shown), or another data connection means known in the art, thereby enabling data sharing there between. While the local service 64 is illustrated as a touch screen, it is to be understood that, e.g., a keyboard and/or a monitor could be employed, alternatively or additionally, as a local service. The various data processing and services may incorporate, via their programming, an alert delivery subsystem for automatically notifying a user and/or manufacturer of repeated use and/or excessive wear of the electrosurgical needle 12.

For example, an alert delivery subsystem may be used to issue an alert or notification by changing the active color at the display 30 on the handheld electrosurgical generator 10. Other feedback may be issued at the handheld electrosurgical generator 10 based on output received from the microchip 42 and processing of that output at the generator 10 (or other system processing device 54, 62).

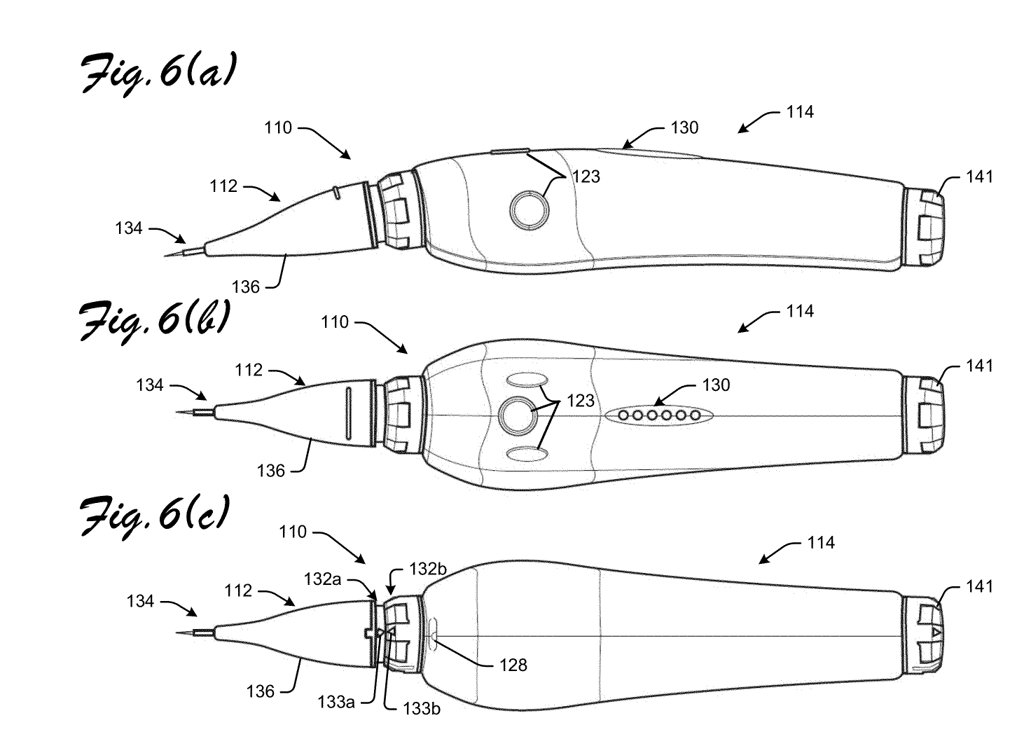

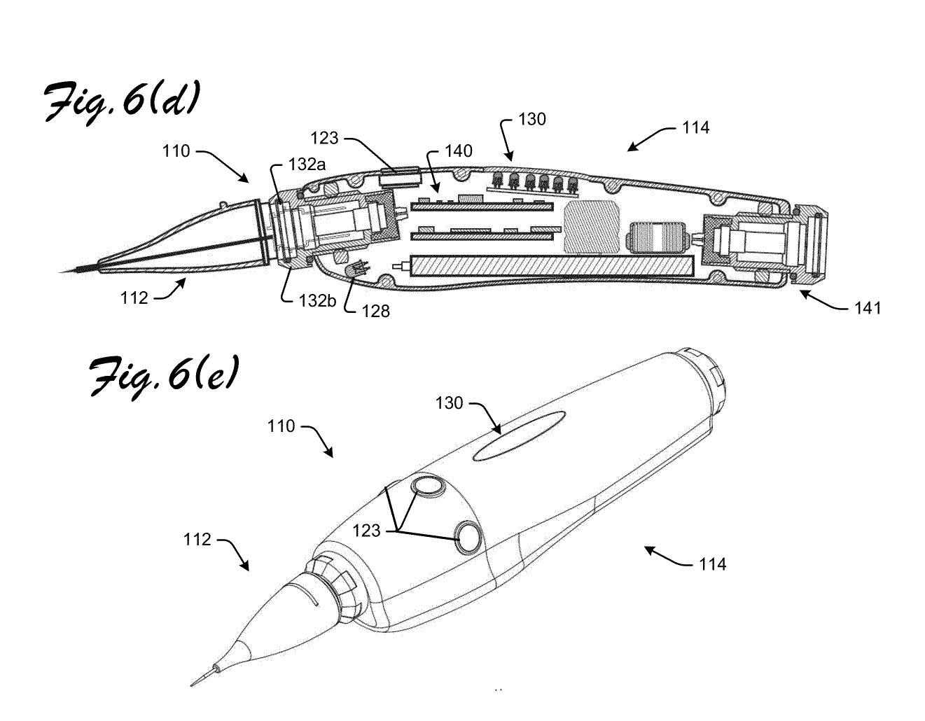

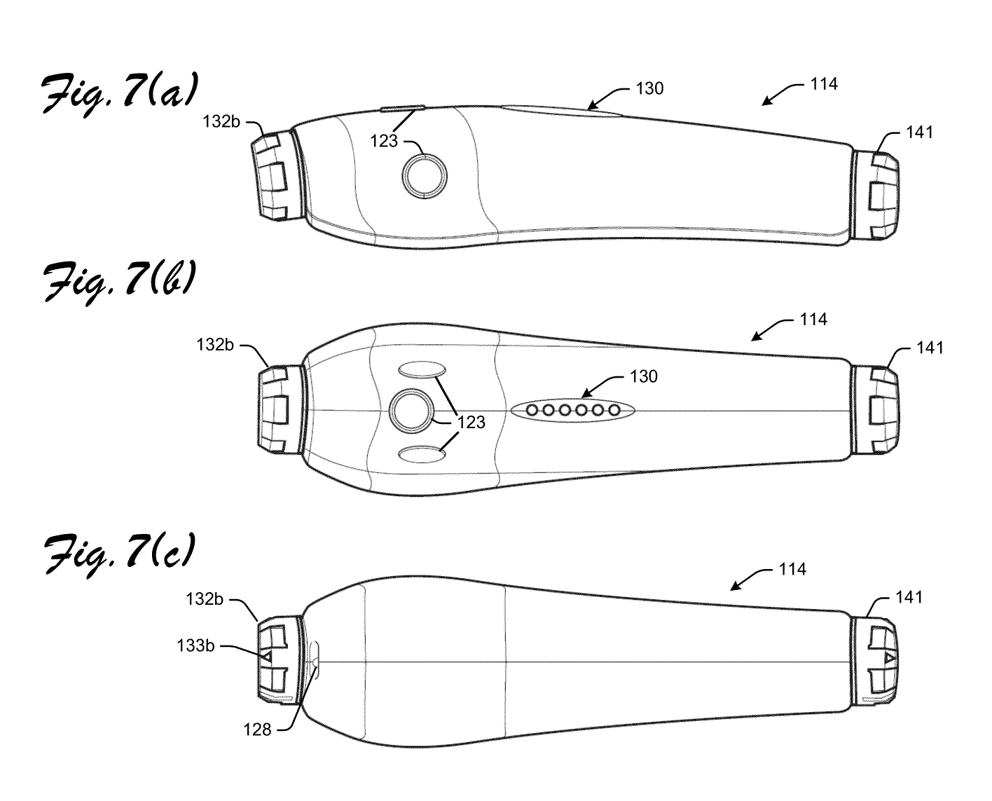

FIG. 6 illustrates an example handheld electrosurgical generator 110 generally including an electrosurgical needle 112 and a pen or handle 114, wherein (a) is a side plan view, (b) is a top plan view, (c) is a bottom plan view, (d) is cross-sectional view, and (e) is a perspective view. FIG. 7 shows the pen 114, detached from the electrosurgical needle 112, wherein (a) is a side plan view, (b) is a top plan view, and (c) is a bottom plan view thereof. It is to be noted that similarly numbered parts (e.g., electrosurgical needle 12, 112) are configured and operate in a similar manner, unless otherwise expressly stated. The electrosurgical needle 112 includes a needle tip 134 and a shroud 136. Meanwhile, the pen or handle 114 includes a series of control elements 123 (e.g., buttons/knobs/sliders) for power and waveform adjustment, a light 128, a display 130, a main controller 140, and a rear electrical connector 141 to permit connection to an outside power and/or data processing source (not shown). It is to be understood that the power source could be, for example, in the form of one or more batteries or an electrical outlet. The electrosurgical needle 112 and the pen 114 together define an electro-mechanical interconnect 132 a-b, with a pair of matching alignment arrows 133 a-b to ensure the appropriate engagement of the two portions of the electro-mechanical interconnect 132 a-b.

In another example, the email notification, e.g., issued by a monitoring service may be used to automatically order new needles 12 and/or batteries 20. The email may be delivered to the user and/or manufacturer. Still other examples of using the handheld electrosurgical generator 10 as part of a larger system (e.g., a full-service surgical center) are also contemplated, as will be understood by those having ordinary skill in the art after becoming familiar with the teachings herein.

It is noted that the examples shown and described are provided for purposes of illustration and are not intended to be limiting. Still other examples are also contemplated.

1. A handheld electrosurgical generator comprising:
a pen having a mechanical interconnect to attach an electrosurgical needle;
a contact associated with the pen to provide an electrical connection between the pen and the electrosurgical needle; and
a controller embedded in the pen and associated with the contact to control electrical output at the electrosurgical needle.

2. The handheld electrosurgical generator of claim 1, further comprising a power source associated with the contact to provide electrical power to the electrosurgical needle.

3. The handheld electrosurgical generator of claim 2, wherein the power source outputs about 3-12 volts DC.

4. The handheld electrosurgical generator of claim 2, wherein the power source is exterior of the pen.

5. The handheld electrosurgical generator of claim 4, wherein the power source includes a disposable electrosurgical grounding pad with an integrated battery and is connected to the pen by a power cable.

6. The handheld electrosurgical generator of claim 5, wherein the integrated battery has a positive terminal on one side of a body patch and a negative terminal on the opposite side of the body patch, the negative side of the body patch for affixing to a patient's body to make a good grounding contact.

7. The handheld electrosurgical generator of claim 6, further comprising at least one of an adhesive and an electrolytic gel to affix the body patch to the patient's body.

8. The handheld electrosurgical generator of claim 1, further comprising at least one control on the pen for a user to adjust at least one of a power level and a waveform output by the electrosurgical needle.

9. The handheld generator of claim 1, further comprising at least one feedback circuit including a body patch and a processor, the at least one feedback circuit providing control of power level and waveform output by the electrosurgical needle based on a detected condition at the body patch.

10. The handheld electrosurgical generator of claim 1, further comprising a display on the pen to provide visual feedback to a user concerning at least one of a power level and a waveform output at the electrosurgical needle.

11. The handheld electrosurgical generator of claim 1, wherein the controller has updateable firmware to provide at least one of a new power and waveform output at the electrosurgical needle.

12. The handheld electrosurgical generator of claim 1, further comprising a microchip to track use of the electrosurgical needle.

13. An electrosurgical system comprising:
a handheld electrosurgical generator comprising:
a pen to electro-mechanically connect to an electrosurgical needle; and
a controller embedded in the pen to control an electrical output at the electrosurgical needle; and
at least one of a data processing unit and a data entry point in communication with the controller.

14. The electrosurgical system of claim 13 wherein the handheld electrosurgical generator and the at least one of a data processing unit and a data entry point are linked to one another by at least one of a network, a wireless connection, and a hard-wired connection.

15. The electrosurgical system of claim 13 wherein the at least one of a data processing unit and a data entry point includes an element chosen from the group consisting of a local data processor, an external processor, and a touch screen.

16. The electrosurgical system of claim 13 wherein the at least one of a data processing unit and a data entry point includes an alert delivery subsystem for automatically notifying a user and/or manufacturer of repeated use and/or excessive wear of the electrosurgical needle.

17. The electrosurgical system of claim 13 wherein the at least one of a data processing unit and a data entry point is either local or remote.

18. A handheld electrosurgical generator comprising:
a pen to electro-mechanically connect with an electrosurgical needle; and
a controller embedded within the pen to control an electrical output at the electrosurgical needle.

19. The handheld electrosurgical generator of claim 18 further comprising at least one control carried by the pen and operatively connected to the controller, the control being configured for allowing manual adjustment of at least one of a power level and a waveform output of the electrical output at the electrosurgical needle.

20. The handheld electrosurgical generator of claim 18 further comprising a power source associated with the pen to provide electrical power to the electrosurgical needle.

21. The handheld electrosurgical generator of claim 18 further comprising a display on the pen to provide visual feedback to a user concerning at least one of a power level and a waveform output at the electrosurgical needle.

22. The handheld electrosurgical generator of claim 18 further comprising a audio transducer within the pen to provide audible feedback to a user concerning at least one of a power level and a waveform output at the electrosurgical needle.

23. The handheld electrosurgical generator of claim 18 wherein the generator operates in monopolar mode.

24. The handheld electrosurgical generator of claim 18 wherein the generator operates in bipolar mode.
